(19) United States
(12) Patent Application Publication
(10) Pub. No.: US 2005/0288187 A1
(43) Pub. Date: Dec. 29, 2005

(54) Inhibitor development for 4-hydroxyphenylpyruvate dioxygenase, employing tyrosinemia 1 as a model for human diseases mediated by 2-oxoacid utilizing dioxygenases

(76) Inventors: Hartmut M. Hanauske-Abel, Englewood Cliffs, NJ (US); Anthony Popowicz, Farmingville, NY (US)

Correspondence Address: Michael L Goldman;Nixon Peabody, Clinton Square, PO Box 31051, Rochester, NY, 14603, US

(21) Appl. No.: 10/519,370

(22) PCT Filed: Jul. 2, 2003
(86) PCT No.: PCT/US03/21178

§ 371 Date: Jul. 25, 2005

Related U.S. Application Data
(60) Provisional application No. 60/394,339, filed on Jul. 3, 2002.

(51) Int. Cl. A01N 43/40; A61K 31/44

(52) U.S. Cl. 504/254; 514/348

(57) ABSTRACT

The present invention is directed to a method of inhibiting 4-hydroxyphenylpyruvate dioxygenase in a living system by administering to the living system an effective amount of a compound of formulas I or II or III or derivatives thereof as follows: R¹, R², R³, and R⁴ each individually represent a hydrogen, an alkyl, alkenyl, or alkoxy group containing 1 to about 8 carbon atoms, an aryl, aralkyl, or cycloalkyl group containing about 5 to 12 carbon atoms, or a carboalkoxy or carbamyl group containing up to 8 carbon atoms, or a peptide or peptidomimetic moiety containing 10 to about 30 carbon atoms under conditions effective to inhibit 4-hydroxyphenylpyruvate dioxygenase in the living system. These compounds are also useful in carrying out a method of treating a patient and a method of regulating plant growth.

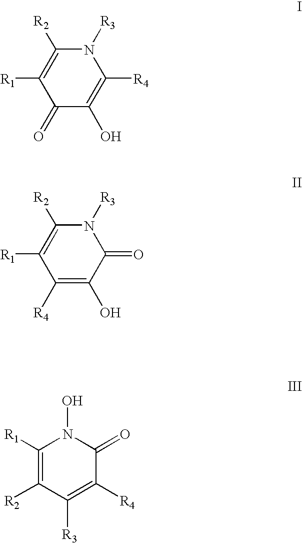

The present invention was made with funding from National Institutes of Health Grant No. RR06020. The United States Government may have certain rights.

### FIELD OF THE INVENTION

The present invention is directed to methods of inhibiting 4-hydroxyphenylpyruvate dioxygenase in a living system, treating a patient with tyrosinemia, and regulating plant growth.

### BACKGROUND OF THE INVENTION

Tyrosinemia I, a recessively inherited disease that profoundly disrupts liver and kidney function, is caused by the deficiency of fumarylacetoacetate hydrolase (EC 3.7.1.2). The pathogenesis of potentially lethal liver damage in tyrosinemia I involves, however, two additional enzymes: 4-hydroxyphenylpyruvate dioxygenase (4-HPPD; EC 1.13.11.27), which forms the actual hepatotoxins; and prolyl 4-hydroxylase (P-4H; E.C. 1.14.11.2), required for the reactive hepatic fibrosis and indispensable for collagen synthesis in general. 4-HPPD and P4-H are non-heme iron enzymes, and each consumes one molecule of atmospheric oxygen and one 2-oxoacid moiety while hydroxylating one carbon atom. 4-HPPD and P-4H have long been classified together as 2-oxoacid-utilizing dioxygenases (Abbott et al., “Alpha-Ketoglutarate-Coupled Dioxygenases,” in Hayaishi, ed. Molecular Mechanisms of Oxygen Activation, New York: Academic Press, pp. 167-214 (1974), and, therefore, tyrosinemia I ranks as a model for human diseases mediated by this class of enzymes, diseases that prominently include all fibrotic conditions. A single, collective catalytic pathway—the ‘HAG’ mechanism, proposed in 1982 by Hanauske-Abel and Günzler—resolves the substrate alignment, catalytic orbital interactions, and product release of the 2-oxoacid-utilizing dioxygenases (Hanauske-Abel et al., “A Stereochemical Concept for the Catalytic Mechanism of Prolyl Hydroxylase. Applicability to Classification and Design of Inhibitors,” J. Theor. Biol. 94:421-55 (1982); Hanauske-Abel, “Über einen Stereochemischen Vorschlag für den katalytischen Mechanismus der Prolylhydroxylase, seine Anwendung zur Klassifizierung und Formulierung von Hemmstoffen sowie die Entwicklung und Erprobung eines ‘maβgeschneiderten’ neuartigen Inhibitors,” Department of Pharmacol. Toxicol., Philipps University, Marburg an der Lahn, Germany, M.D., Ph.D., Thesis (1983); Hanauske-Abel, “Fibrosis: Representative Molecular Elements, A Basic Concept, and Emerging Targets for Suppressive Treatment,” in Zakim, eds. Hepatology, A Textbook of Liver Disease,” Philadelphia: W.B. Saunders, pp. 465-506 (1996), and has been extremely useful for the rational, often computer-assisted discovery of inhibitors and candidate drugs targeting these enzymes (Hanauske-Abel et al., “The HAG Mechanism: A Molecular Rationale for the Therapeutic Application of Iron Chelators in Human Diseases Involving the 2-Oxoacid Utilizing Dioxygenases,” Curr. Med. Chem. 10:1005-19 (2003); Hanauske-Abel, “Prolyl 4-Hydroxylase, A Target Enzyme for Drug Development. Design of Suppressive Agents and the In Vitro Effects of Inhibitors and Proinhibitors,” J. Hepatol. 13(Suppl. 3):S8-S16 (1991); Günzler et al., “Prolyl 4-Hydroxylase Inhibitors,” in Guzman N A ed., Prolyl Hyroxylase, Protein Disulfide Isomerase, and Other Structurally Related Proteins, New York: Decker, pp. 65-96 (1997). As expected, the HAG mechanism-utilizing enzymes display a remarkable cross-species susceptibility to such inhibitors; e.g., human and plant 4-HPPD are similarly sensitive to the cyclohexanetrione class of plant growth retardants (Schulz et al., “SC-0051, A 2-Benzoyl-Cyclohexane-1,3-Dione Bleaching Herbicide, is a Potent Inhibitor of the Enzyme P-Hydroxyphenylpyruvate Dioxygenase,” FEBS Lett. 318:162-166 (1993); Ellis et al., “Inhibition of 4-Hydroxyphenylpyruvate Di-Oxygenase by 2-(2-Nitro-4-Trifluormethylbenzoyl)-Cyclohexane-1,3-Dione and by 2-(2-Chloro-4-Methanesulfonylbenzoyl)-Cyclohexane-1,3-Dione,” Toxicol. Appl. Pharmacol. 133:12-19 (1995). The cyclohexanetrione herbicides also inhibit other 2-oxoacid-utilizing dioxygenases in plants, such as the hydroxylases essential for synthesis of gibberellin hormones (Nakayama et al., “Effect of a Plant-Growth Regulator, Prohexadione, on the Biosynthesis of Gibberellins in Cell-Free Systems Derived from Immature Seeds, Plant Cell Physiol. 31:1183-90 (1990); Rademacher, “Biochemical Effects of Plant Growth Retardants,” in Gausmann, ed., Plant Biochemical Regulators, New York: Dekker, pp. 169-200 (1991). Like human P-4H, the gibberellin hydroxylases use the 2-oxoacid moiety of 2-oxoglutarate (Hanauske-Abel, “Fibrosis: Representative Molecular Elements, A Basic Concept, and Emerging Targets for Suppressive Treatment,” in Zakim, eds. Hepatology, A Textbook of Liver Disease,” Philadelphia: W.B. Saunders, pp. 465-506 (1996).

In children with tyrosinemia I, one of these herbicides is used therapeutically to inhibit 4-HPPD, thus suppressing formation of the hepatotoxins maleylacetoacetate, fumarylacetoacetate, and their saturated derivatives (Lindstedt et al., “Treatment of Hereditary Tyrosinemia Type I by Inhibition of 4-Hydroxyphenylpyruvate Dioxygenase,” Lancet 340:813-17 (1992)). Introduced in 1992, this agent, 2-2(-nitro-4-trifluoromethylbenzoyl)-cyclohexane-1,3-dione or NTBC (Orfadin™; Swedish Orphan International AB, Harpenden/United Kingdom), has benefited several hundred tyrosinemia I patients and has changed at least the time frame for liver transplantation, presently still the definitive treatment (Wappner, “Disorders of Amino Acid and Organic Acid Metabolism,” in McMillan, eds., Oski's Pediatrics, Philadelphia: Lippincott, pp. 1828-1847 (1999); Holme et al., “Tyrosinemia Type I and NTBC [2-(2-Nitro-4-Trifluoromethylbenzoyl)-1,3-Cyclohexanedione],” J. Inherit Metab. Dis., 21:507-17 (1998). However, no specific consideration was given to any inhibitory effect NTBC might exert, or might fail to exert, on other 2-oxoacid-utilizing dioxygenases in humans, and its mode of binding to 4-HPPD, the basis for its clinical utility, also has received little attention.

The present invention is directed to overcoming these deficiencies in the art.

### SUMMARY OF THE INVENTION

The present invention is directed to a method of inhibiting 4-hydroxyphenylpyruvate dioxygenase in a living system by administering to the living system an effective amount of a compound of formulas I or II or III or derivatives thereof as follows: 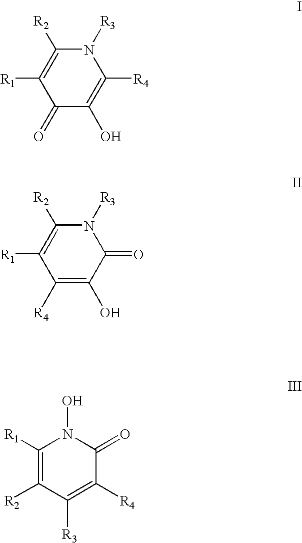
R¹, R², R³, and R⁴ each individually represent a hydrogen, an alkyl, alkenyl, or alkoxy group containing 1 to about 8 carbon atoms, an aryl, aralkyl, or cycloalkyl group containing about 5 to 12 carbon atoms, or a carboalkoxy or carbamyl group containing up to 8 carbon atoms, or a peptide or peptidomimetic moiety containing 10 to about 30 carbon atoms under conditions effective to inhibit 4-hydroxyphenylpyruvate dioxygenase in the living system.

Another aspect of the present invention is directed to a method of treating a patient with tyrosinemia by administering to the patient an effective amount of a compound of formulas I or II or III or derivatives thereof as follows: 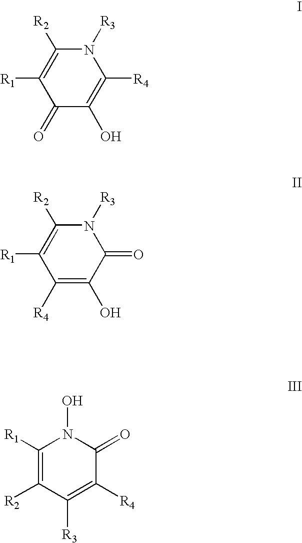
R¹, R², R³, and R⁴ each individually represent a hydrogen, an alkyl, alkenyl, or alkoxy group containing 1 to about 8 carbon atoms, an aryl, aralkyl, or cycloalkyl group containing about 5 to 12 carbon atoms, or a carboalkoxy or carbamyl group containing up to 8 carbon atoms, or a peptide or peptidomimetic moiety containing 10 to about 30 carbon atoms under conditions effective to treat tyrosinemia.

Yet another aspect of the present invention relates to a method of regulating plant growth by administering to a plant an effective amount of a compound of formulas I or II or III or derivatives thereof as follows: 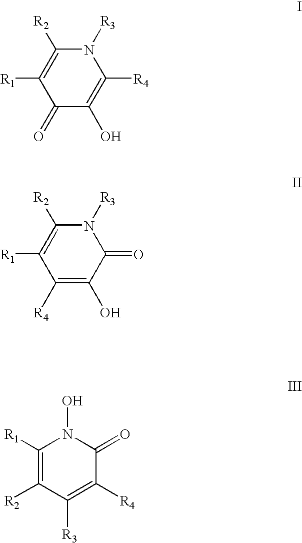
R¹, R², R³, and R⁴ each individually represent a hydrogen, an alkyl, alkenyl, or alkoxy group containing 1 to about 8 carbon atoms, an aryl, aralkyl, or cycloalkyl group containing about 5 to 12 carbon atoms, or a carboalkoxy or carbamyl group containing up to 8 carbon atoms, or a peptide or peptidomimetic moiety containing 10 to about 30 carbon atoms under conditions effective to regulate plant growth.

Applicants have discovered that treatment of tyrosinemia I with NTBC allows the simultaneous assessment of inhibition of 4-HPPD and P4-H in vivo. Surrogate biomarkers for the activity of either enzyme in a patient with established tyrosinemia I are monitored and prospectively measured for the first 100 days of NTBC administration. Only the 4-HPPD biomarkers normalized. These empirical observations are due to a general biologic factor—i.e., the distinct difference between the active site topography of 4-HPPD and of P4-H.

### BRIEF DESCRIPTION OF THE DRAWINGS

FIG. 1 shows the effect of NTBC. The shaded area indicates the range of normals, the hatched bar the NTBC treatment period. Biomarkers for 4-HPPD activity: Plasma succinylacetone (large open circles; normal: <0.1 μmol/L) and urine succinylacetoacetate (small open circles; normal: <1.0 mmol/mol creatinine). Biomarkers for P4-H activity; PIIINP, indicating collagen type III (solid squares; normal: <0.8 U/ml); NCl, indicating collagen type IV (solid circles; normal: <9.5 ng/ml); and NTD, indicating collagen type VI (solid triangles; normal: <530 ng/ml). Control biomarker for P4-H activity: P1, derived from laminin (open triangles; normal: <1.8 ng/ml).

FIGS. 2A-B show the structure and binding of NTBC. FIG. 2A shows the structure of NTBC. The domains shared between 4-hydroxyphenylpyruvate (4-HPP), the substrate of 4-HPPD, and the drug 2-(2-nitro-4-trifluoromethylbenzoyl)-cyclohexane-1,3-dione (NTBC) are highlighted (I, benzene ring; II, 2-oxoacid group or its analog). NTBC displays an acetylacetone moiety that preferentially tautomerizes into its keto-enol form (Rademacher, “Biochemical Effects of Plant Growth Retardants,” in Gausmann, ed., Plant Biochemical Regulators, New York: Dekker, pp. 169-200 (1991), which are hereby incorporated by reference in their entirety), enabling interaction with the non-heme iron atom (Fe) at the active site of 4-HPPD, 4-HPP binds via bidentate, NTBC via terdentate coordination. FIG. 2B shows the binding of NTBC to 4-HPPD. The steric arrangement of the ligands NTBC (4) and 4-HPP (3) at the non-heme iron atom (1) in the active site pocket of 4-HPPD is depicted. The peptide backbone of the enzyme, shown as gray ribbon, has been cut open for visualization. Two histidine (5a) and one carboxylate (5b) side chain coordinate the iron atom to the apoprotein. This non-heme iron atom displays the typical ‘bipolar’, cisoid alignment of reactants as required by the HAG mechanism (Hanauske-Abel et al., “A Stereochemical Concept for the Catalytic Mechanism of Prolyl Hydroxylase. Applicability to Classification and Design of Inhibitors,” J. Theor. Biol. 94:421-55 (1982); Hanauske-Abel et al., “The HAG Mechanism: A Molecular Rationale for the Therapeutic Application of Iron Chelators in Human Diseases Involving the 2-Oxoacid Utilizing Dioxygenases,” Curr. Med. Chem. 10:1005-19 (2003); and Hanauske-Abel, “Fibrosis: Representative Molecular Elements, A Basic Concept, and Emerging Targets for Suppressive Treatment,” in Zakim, eds. Hepatology, A Textbook of Liver Disease,” Philadelphia: W.B. Saunders, pp. 465-506 (1996), which are hereby incorporated by reference in their entirety): apoenzyme residues on one half-sphere, and substrates or inhibitors on the other half-sphere. The O₂-derived dioxygen substrate is shown broken open in eggshell fashion (2) to display the position occupied by the nitro group of NTBC. The appenzyme residues that hold the non-heme iron atom, identified for bacterial and, in italics, for human 4-HPPD, occur in highly conserved segments (73% identity/similarity by ClustalW formatted alignment of the bacterial and human sequences: accession numbers of 1CJXA and AAC73008, respectively). Note that in NTBC as well as in the substrate 4-HPP, the planes of the shared domains (I. and II.) stand almost perpendicular on each other.

FIG. 3 shows the binding of 1,2-dimethyl-3-hydroxypyrid-4-one (DMHP), a representative compound of the present invention and also a known inhibitor of prolyl 4-hydroxylase and thus collagen biosynthesis (U.S. Pat. Nos. 5,789,426; 5,965,585; and 5,965,586, which are hereby incorporated by reference in their entirety) to the active site of 4-HPPD. The steric arrangement of the ligands, either the substrate 4-HPP (3) or the inhibitor DMPH (4), at the non-heme iron atom (1) in the active site pocket of 4-HPPD is depicted. The iron-bound dioxygen is highlighted (2), as are the residues involved in binding of the metal atom (5a and 5b) or of the substrate (5c). Thus, in contrast to NTBC, DMHP can access and block the active site of prolyl 4-hydroxylase as well as of 4-HPPD.

### DETAILED DESCRIPTION OF THE INVENTION

The present invention is directed to a method of inhibiting 4-hydroxyphenylpyruvate dioxygenase in a living system by administering to the living system an effective amount of a compound of formulas I or II or III or derivatives thereof as follows: 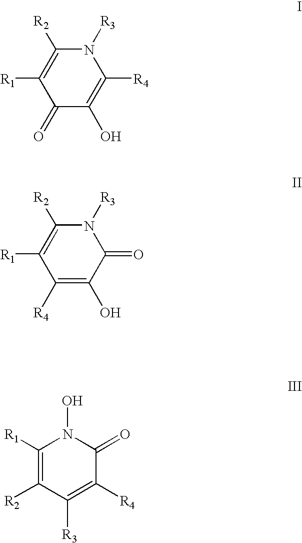
R¹, R², R³, and R⁴ each individually represent a hydrogen, an alkyl, alkenyl, or alkoxy group containing 1 to about 8 carbon atoms, an aryl, aralkyl, or cycloalkyl group containing about 5 to 12 carbon atoms, or a carboalkoxy or carbamyl group containing up to 8 carbon atoms, or a peptide or peptidomimetic moiety containing 10 to about 30 carbon atoms under conditions effective to inhibit 4-hydroxyphenylpyruvate dioxygenase in the living system.

The alkyl, alkenyl, alkoxy, aryl, aralkyl, and cycloalkyl groups represented by R₁, R₂, R₃, and R₄ can be substituted or unsubstituted. Examples of unsubstituted alkyl groups are methyl, ethyl, propyl, isopropyl, butyl, sec-butyl, pentyl, hexyl, and the like. Unsubstituted alkenyl groups can be 2-propenyl, 2-butenyl, 3-butenyl, 2-methyl-2-butenyl, and the like. Unsubstituted alkoxy groups can be methoxy, ethoxy, propoxy, sopropoxy, and the like. Unsubstituted aryl groups can be phenyl or naphthyl. Aralkyl groups can be, for example, benzyl and phenylethyl. Cycloalkyl groups can be cyclopentyl, cyclohexyl, 4-methyl cyclohexyl, and the like. For substituted alkyl, alkenyl, alkoxy, aryl, aralkyl, and cycloalkyl groups, substituents can include, for example, halo, alkoxy, amino, hydroxy, carboxy, carboalkoxy, and carbamyl. Aryl and aralkyl groups can, in addition, contain alkyl substituents.

Table I contains representative forms of the hydroxypyridone compounds of Formula (I) of the present invention:

TABLE I

| | Trivial name | R₁ | R₂ | R₃ | R₄ |
|---|---|---|---|---|---|
| I | L-mimosine | —CH₂CH(COOH)NH₂ | H | H | H |
| | HK-1, CP20, L1, DMHP | —CH₃ | —CH₃ | H | H |
| | HK-2, CP94 | —CH₂CH₃ | —CH₂CH₃ | H | H |
| | CP93 | —CH₃ | —CH₂CH₃ | H | H |
| | CP96 | —(CH₂)₂OCH₃ | —CH₂CH₃ | H | H |
| | HK-26, CP21 | —CH₂CH₃ | —CH₃ | H | H |
| | HK-27, CP22 | —(CH₂)₂CH₃ | —CH₃ | H | H |
| | HK-16 | —CH₂CH═CH₂ | —CH₃ | H | H |
| | CP23 | —CH₂(CH₃)₂ | —CH₃ | H | H |
| | CP40 | —(CH₂)₂OH | —CH₃ | H | H |
| | CP41 | —(CH₂)₃OH | —CH₃ | H | H |
| | CP42 | —(CH₂)₄OH | —CH₃ | H | H |
| | CP43 | —(CH₂)₅OH | —CH₃ | H | H |
| | CP44 | —(CH₂)₂NH₂ | —CH₃ | H | H |
| | HK-15, CP51 | —(CH₂)₂OCH₃ | —CH₃ | H | H |
| | CP54 | —CH(CH₃)CH₂OCH₃ | —CH₃ | H | H |
| | CP52 | —(CH₃)₃OCH₂CH₃ | —CH₃ | H | H |
| | | —CH₃ | H | —CH₃ | H |
| | | —CH₃ | H | H | —CH₃ |
| | | — | H | H | H |
| | | CH₂CH(COOC₂H₅)NH₂ | | | |

Table II contains representative structures of the hydroxypyridone compounds of Formula (II) of the present invention:

TABLE II

| | R₁ | R₂ | R₃ | R₄ |
|---|---|---|---|---|
| II | —CH₃ | —CH₃ | H | H |
| | —CH₂CH₃ | —CH₂CH₃ | H | H |
| | —CH₃ | —CH₂CH₃ | H | H |
| | —(CH₂)₂OCH₃ | —CH₂CH₃ | H | H |
| | —CH₂CH₃ | —CH₃ | H | H |
| | —(CH₂)₂CH₃ | —CH₃ | H | H |
| | —CH₃ | H | —CH₃ | H |
| | —CH₃ | H | H | —CH₃ |

Compounds of Formula (I)

Compounds of Formula (I) are synthesized by one of several general procedures.

Method A

This method is adapted from Kontoghiorghes and Sheppard, Inorg. Chim. Acta 136:L11-L12 (1987), which is hereby incorporated by reference in its entirety. In brief, a 3-hydroxy-4-pyrone is refluxed for approximately six hours with three equivalents of a primary amine dissolved in an appropriate solvent. The reaction mixture is decolorized with charcoal, filtered, and the filtrate evaporated to give a dark residue. The residue is recrystallized from one to three times from an appropriate solvent to yield a solid product with a narrow melting point and an NMR spectrum consistent with the structure anticipated.

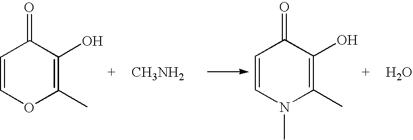

In particular, 3-hydroxy-2-methyl-4-pyrone (10 g) was refluxed for 6.5 hours with three equivalents of aqueous methylamine (40%) in 200 ml of water. The reaction mixture was allowed to cool after which decolorizing charcoal was added to the solution, and the mixture was stirred for 0.5 hours. After filtration, the solvent was evaporated under reduced pressure, and the solid residue recrystallized three times from water to yield 1,2-dimethyl-3-hydroxypyrid-4-one (HK-1, L1, CP20, DMHP) as fine white needles.

In a slight modification of this procedure, N-carboxymethyl-3-hydroxy-2-methylpyrid-4-one is prepared as described by Zhang et al., Can. J. Chem. 70:763-770 (1992), which is hereby incorporated by reference in its entirety. One equivalent of 3-hydroxy-2-methyl-4-pyrone and two equivalents of glycine are dissolved in hot distilled water, the pH is adjusted to approximately 9 with 8 N sodium hydroxide, and the reaction mixture is heated under reflux for 20 hours. After cooling and decolorizing with charcoal, approximately half of the solvent is removed under vacuum and 6 N hydrochloric acid is added to reduce the pH to approximately 3. A yellow solid precipitates which yields the product as off-white crystals after two recrystallizations from water (mp 258-260 C).

Method B

This method is adapted from GB Patent No. 2,118,176A to Hider et al., which is hereby incorporated by reference in its entirety. In brief, a 3-hydroxy-4-pyrone is converted to the corresponding 3-benzyloxy-4-pyrone via reaction with benzyl chloride. A methanolic solution of the pyrone is added to an aqueous solution of sodium hydroxide after which benzyl chloride is added and the reaction mixture refluxed for approximately six hours. The solvent is evaporated under reduced pressure, water is added, and then the product is extracted into an appropriate organic solvent. After washing, the extract is dried over anhydrous magnesium sulfate and the solvent evaporated to yield the crude 3-benzyloxy derivative which is used in the next step without further purification. To a solution of the 3-benzyloxy compound in an appropriate solvent is added a slight excess of primary amine. The reaction mixture is stirred at room temperature for approximately six days after which it is acidified to pH 2 with concentrated hydrochloric acid and evaporated to dryness. The residue is washed with water and extracted into an appropriate organic solvent which is then dried over magnesium sulfate and evaporated to dryness. To the residue is added hydrobromic acid. This reaction mixture is heated on a steam bath for 30 minutes and then recrystallized from water to yield the N-substituted 3-hydroxypyrid-4-one. The product melts sharply and has an NMR spectrum consistent with the desired product. This synthesis is depicted as follows:

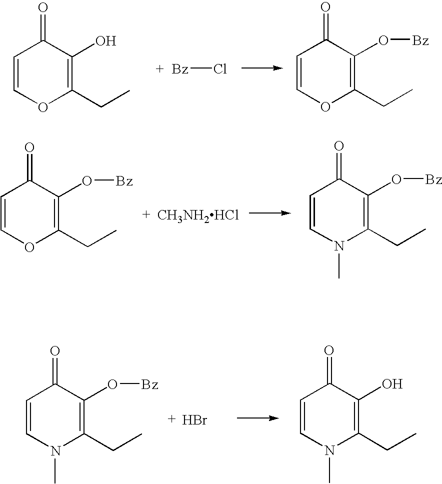

In particular, 2-ethyl-3-hydroxy-4-pyrone (24.7 g) in 225 ml of methanol is added to 25 ml of water containing 7.5 g of sodium hydroxide. To this solution is added benzyl chloride (25.5 g) and the mixture is then refluxed for six hours. Upon cooling, the solvent is removed under reduced pressure. The residue is treated with 50 ml of water and then extracted three times with 25-ml aliquots of dichloromethane. The combined extracts are washed twice with 5% (w/v) sodium hydroxide, then twice with 25 ml of water and dried over magnesium sulfate. Evaporation of the solvent yields crude 3-benzyloxy-2-ethyl-4-pyrone. This crude pyrone (24.4 g) and 1.56 g of methylamine hydrochloride are then dissolved in 300 ml of aqueous ethanol (100 ml) containing 2 g of sodium hydroxide. The solution is stirred at room temperature for six days, acidified to pH 2 with concentrated hydrochloric acid, and then evaporated to dryness. The residue is washed with water and extracted twice into chloroform (50 ml). The combined extracts are dried over anhydrous magnesium sulfate and evaporated to dryness yielding 3-benzyloxy-2-ethyl-1-methylpyrid-4-one. To 2 g of this pyrid-4-one is added concentrated hydrobromic acid (10 ml). The reaction mixture is heated on a steam bath for 30 minutes, and the product is recrystallized from water to yield 2-ethyl-3-hydroxy-1-methylpyrid-4-one.

Method C

This method is adapted from that of Bartulin et al., J. Heterocyclic Chem. 29:1017-1019 (1992), which is hereby incorporated by reference in its entirety. A 3-benzyloxy-4-pyrone, prepared as in Method B, is added to an ethanolic solution of aqueous ammonia. The reaction mixture is stirred for approximately three days, concentrated under reduced pressure, triturated with acetone, and the solid recrystallized from ethanol to yield the corresponding 3-benzyloxypyrid-4-one. To a solution of this pyrid-4-one in aqueous ethanol containing one equivalent of sodium hydroxide was added an equivalent of n-alkyl bromide. The reaction mixture was heated under reflux for 24 hours after which it was cooled, concentrated under reduced pressure, and extracted with an appropriate solvent. After washing, the organic phase with water, it is dried over magnesium sulfate. The product is obtained upon concentration of the solution under reduced pressure. Crude 1-alkyl-3-benzyloxypyrid-4-one in acetic acid containing 40% hydrobromic acid is then heated on a steam bath for 30 minutes. The 1-alkyl-3-hydroxypyrid-4-one precipitates and is subsequently recrystallized from benzene in good yield with a narrow melting point and appropriate NMR spectrum. This is shown in the following synthesis:

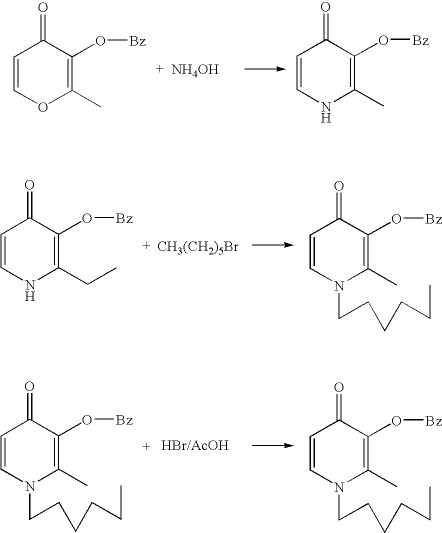

In particular, 3-benzyloxy-2-methyl-4-pyrone was prepared as described in Method B. A solution containing 15.3 g of the pyrone, 160 ml of aqueous ammonia (25%), and 80 ml of ethanol is stirred at room temperature for three days. The solvent is removed under reduced pressure and some acetone is added. The solid which precipitates is collected by filtration and recrystallized from ethanol to yield (80%) 3-benzyloxy-2-methylpyrid-4-one with a melting point of 162-163 C. A solution containing 0.125 moles of the pyrid-4-one, 0.125 moles of n-hexyl bromide, 0.125 moles of sodium hydroxide, 25 ml of water, and 200 ml of ethanol is heated under reflux for 24 hours. After removal of the solvent under vacuum, the residue is extracted with ethyl ether. The etherial solution is washed with water yielding a precipitate which is crystallized from benzene after drying to give 3-benzyloxy-1-hexyl-2-methylpyrid-4-one (95%, mp 46° C.). A solution of this compound in 80 ml of acetic acid containing 40% hydrobromic acid is then heated on a steam bath for 30 minutes. The product is filtered off and crystallized from benzene to yield 1-hexyl-3-hydroxy-2-methylpyrid-4-one in 70% yield.

Compounds of Formula (II)

Compounds of Formula (II) are synthesized by the general procedure outlined in GB Patent No. 1,118,176A to Hider et al., which is hereby incorporated by reference in its entirety. In brief, 2,3-dihydroxypyridine is mixed with an organic halide in a sealed tube and heated at 140° C. for 24 hours. The tube is then cooled in an acetone/dry ice bath and opened. The excess halide is poured off, and water is added to the dark residue. Sulfur dioxide gas is bubbled through the mixture until the aqueous phase becomes clear. The pH of the reaction mixture is then adjusted to approximately 6 with sodium carbonate, and the resulting solution is extracted with an appropriate solvent after saturation with ammonium sulfate. The organic extracts are dried over anhydrous sodium sulfate and concentrated under reduced pressure to yield a solid which gives the desired N-substituted 3-hydroxypyrid-2-one after crystallization from petroleum ether.

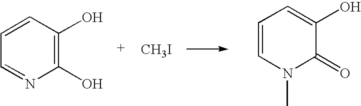

In particular, 5.6 g of 2,3-dihydroxypyridine in 20 ml of methyl iodide are heated in a sealed tube at 140° C. for 24 hours. The tube is cooled in acetone/dry ice, opened, and the excess methyl iodide poured off. Distilled water (10 ml) is added, and the solution is treated with sulfur dioxide until clear. The pH of the reaction mixture is adjusted to 6 with aqueous sodium carbonate (1 M) after which the resulting solution is saturated with ammonium sulfate followed by extraction with chloroform until the chloroform layer fails to give a blue color with aqueous ferric chloride. The combined extracts are dried over sodium sulfate after which the solvent is removed under reduced pressure and the residue crystallized from petroleum ether to give 3-hydroxy-1-methylpyrid-2-one.

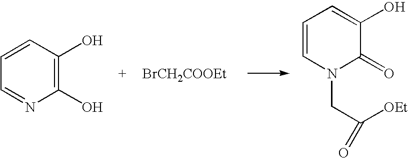

A related compound, 1-ethoxycarbonylmethyl-3-hydroxypyrid-2-one, is prepared by heating a mixture of 2,3-dihydroxypyridine (5 g) and 20 ml of ethylbromoacetate in a sealed tube at 140° C. for 24 hours, as described by GB Patent No. 4,585,780 to Hider et al., which is hereby incorporated by reference in its entirety. After cooling in solid CO₂, the tube is opened, the reaction mixture poured off, and evaporated to dryness under vacuum to yield a yellow solid. Recrystallization from water yields the product as white crystals (5.4 g), MP 141-151° C.

Compounds of Formula (III)

Compounds of Formula (III) are synthesized by the general procedure outlined in Kellner et al., “Pharmacokinetics and Biotransformation of the Antimycotic Drugs Ciclopiroxolamine In Animals and Man After Topical and Systemic Administration,” Arzneimittelforschung 31:1337-1353 (1981), which is hereby incorporated by reference in its entirety.

The compounds of the present invention can be administered topically or systemically to living beings. More particularly, in humans and animals, such administration can be orally; parenterally, i.e. by subcutaneous, intravascular, or intramuscular injection; intraperitoneally; intrathecally; or by topical application, e.g. to skin or eyes, or by application to the mucous membranes of the nose, throat, bronchial tree, or rectum, etc. They may be administered alone or with suitable pharmaceutical carriers, and can be in solid or liquid form such as tablets, capsules, powders, solutions, suspensions, or emulsions. The dosage of the active compound depends on the species of warm-blooded animal, the body weight, age, and mode of administration.

The pharmaceutical products of the present invention are prepared by dissolving, mixing, granulating, or tablet-coating processes which are known per se.

For oral administration, the active compounds or their physiologically tolerated derivatives such as salts, esters, or amides, are mixed with the additives customary for this purpose, such as vehicles, stabilizers, or inert diluents, and are converted by customary methods into a suitable form for administration, such as tablets, coated tablets, hard or soft gelatin capsules, aqueous, alcoholic, or oily suspensions, or aqueous, alcoholic or oily solutions. Examples of suitable inert vehicles are conventional tablet bases such as lactose, sucrose, or cornstarch in combination with binders like acacia, cornstarch, gelatin, or with disintegrating agents such as cornstarch, potato starch, alginic acid, or with a lubricant like stearic acid or magnesium stearate. Examples of suitable oily vehicles or solvents are vegetable or animal oils, such as sunflower oil or fish-liver oil. Preparations can be effected both as dry and as wet granules.

For parenteral administration (subcutaneous, intravascular, or intramuscular injection), the active compounds or their physiologically tolerated derivatives such as salts, esters, or amides, are converted into a solution, suspension, or emulsion, if desired, with the substances customary and suitable for this purpose, such as solubilizers or other auxiliaries. Examples are: sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solution, and glycols such as propylene glycol or polyethylene glycol, are preferred liquid carriers, particularly for injectable solutions.

For use as aerosols, such as required for the spraying of plants, the active compounds or their derivatives such as salts, esters, or amides, may be dissolved or suspended in an acceptable liquid and packaged in a pressurized aerosol container together with suitable propellants, for example, hydrocarbon propellants like propane, butane, or isobutane with conventional adjuvants. The agents, in accordance with the present invention, may also be administered from a non-pressurized container such as a nebulizer or atomizer.

For topical administration to external or internal body surfaces, e.g., in the form of creams, gels, or drops, etc., the active compounds or their physiologically tolerated derivatives such as, salts, esters, or amides, are prepared and applied as solutions, suspensions, or emulsions in a physiologically acceptable diluent with or without a pharmaceutical carrier.

Another aspect of the present invention is directed to a method of treating a patient with tyrosinemia by administering to the patient an effective amount of a compound of formulas I or II or III or derivatives thereof as follows: 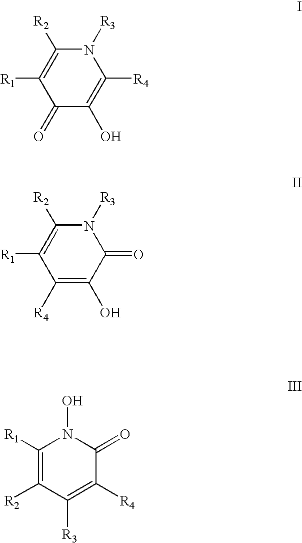
R¹, R², R³, and R⁴ each individually represent a hydrogen, an alkyl, alkenyl, or alkoxy group containing 1 to about 8 carbon atoms, an aryl, aralkyl, or cycloalkyl group containing about 5 to 12 carbon atoms, or a carboalkoxy or carbamyl group containing up to 8 carbon atoms, or a peptide or peptidomimetic moiety containing 10 to about 30 carbon atoms under conditions effective to treat tyrosinemia.

This embodiment of the present invention can be carried out using the compounds and modes of administration described above.

Yet another aspect of the present invention relates to a method of regulating plant growth by administering to a plant an effective amount of a compound of formulas I or II or III or derivatives thereof as follows: 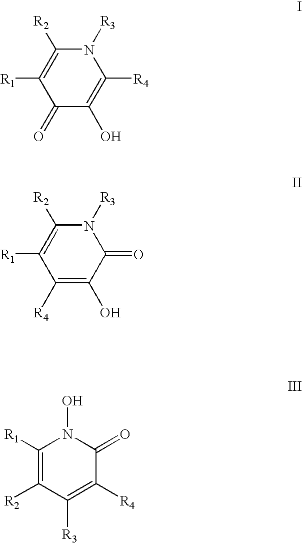
R¹, R², R³, and R⁴ each individually represent a hydrogen, an alkyl, alkenyl, or alkoxy group containing 1 to about 8 carbon atoms, an aryl, aralkyl, or cycloalkyl group containing about 5 to 12 carbon atoms, or a carboalkoxy or carbamyl group containing up to 8 carbon atoms, or a peptide or peptidomimetic moiety containing 10 to about 30 carbon atoms under conditions effective to regulate plant growth.

This embodiment of the present invention can be carried out using the compounds described above. These compounds can be applied to plants or plant seeds, either by topical application such as spraying, or by systemic application via absorption through the root system of a plant when delivered in solubilized form to said system.

Suitable plants include dicots and monocots. More particularly, useful crop plants can include: alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane. Examples of suitable ornamental plants are: Arabidopsis thaliana, Saintpaulia, petunia, pelargonium, poinsettia, chrysanthemum, carnation, and zinnia.

The method of the present invention involving application of the compounds of the present application to plants can be carried out through a variety of procedures when all or part of the plant is treated, including leaves, stems, roots, etc. Suitable application methods include high or low pressure spraying, injection, and leaf abrasion proximate to when application of the compound takes place. When treating plant seeds or propagules (e.g., cuttings), in accordance with the application embodiment of the present invention, the compounds of the present invention can be applied by low or high pressure spraying, coating, immersion, or injection. Other suitable application procedures can be envisioned by those skilled in the art. Plants may be treated with one or more applications of the compounds of the present invention to regulate plant growth.

The compounds of the present invention, can be applied to plants or plant seeds alone or in a mixture with other materials. Alternatively, the compounds can be applied separately to plants with other materials being applied at different times.

A composition suitable for treating plants or plant seeds in accordance with the application embodiment of the present invention contains the compound of the present invention in a carrier. Suitable carriers include water, aqueous solutions, slurries, or dry powders.

Although not required, this composition may contain additional additives including fertilizer, insecticide, fungicide, nematacide, and mixtures thereof. Suitable fertilizers include (NH₄)₂NO₃. An example of a suitable insecticide is Malathion. Useful fungicides include Captan.

Other suitable additives include buffering agents, wetting agents, coating agents, and abrading agents. These materials can be used to facilitate the process of the present invention. In addition, the compounds of the present invention can be applied to plant seeds with other conventional seed formulation and treatment materials, including clays and polysaccharides.

### EXAMPLES

### Example 1

### Experimental Design and Assays Used in the Clinical Investigation

The investigation used IRB-approved protocols that form an integral part of the Children's Clinical Research Center (CCRC) at the Weill Medical College of Cornell University. Measurements of the matrix biomarkers as well as the molecular modeling of 4-hydroxyphenylpyruvate and 2-(2-nitro-4-trifluoromethylbenzoyl)-cyclohexane-1,3-dione were conducted in the Connective Tissue Core Laboratory of the CCRC (Director: H. M. Hanauske-Abel, MD PhD).

A total of ten biomarkers were monitored from just before initiation of NTBC medication to up to 103 days of treatment, generating a total of 60 data points. 4-HPPD activity was assessed by excretion of succinylacetoacetate and succinylacetone, metabolic derivatives of the 4-HPPD product homogentisic acid, and by 4-aminolevulinate in urine. In plasma, methionine and succinylacetone, and in serum α-fetoprotein also were quantified. These indices were determined by standard methods (Lindstedt et al., “Treatment of Hereditary Tyrosinemia Type I by Inhibition of 4-Hydroxyphenylpyruvate Dioxygenase,” Lancet 340:813-17 (1992), which is hereby incorporated by reference in its entirety). P4-H activity was assessed via specific antigens released from three different groups of collagens, all of which are substrates for this enzyme. These biomarkers, quantified by specific radioimmunoassay, consisted of PIIINP, an indicator for the fibril-forming collagen type III; NC1, a marker for the sheet-forming collagen type IV; and NTD, an indicator for the beaded filament-forming collagen type VI. The P1 antigen, derived from the non-collagenous protein laminin and thus not dependent on P4-H activity, served as control.

### Example 2

### Study Subject

In an adopted child with unknown family history, tyrosinemia I was diagnosed at age 4 months. Symptoms at presentation included: failure to thrive, hepatosplenomegaly, rickets, coagulopathy, and Fanconi's syndrome. Because of the coagulopathy, a liver biopsy was not performed at that time. The diagnosis of tyrosinemia I was confirmed by the measurement of fumarylacetoacetate hydrolase (EC 3.7.1.2) activity in blood and by the presence of abnormal levels of tyrosine and tyrosine metabolites in serum and urine. Managed exclusively by nutritional therapy, the patient developed along the 25^{th} percentile for weight and the 5^{th} percentile for height, but experienced a tyrosinemic neurologic crisis at age 3 years, with severe muscle weakness in the lower extremities and areflexia. Orfadin™ treatment commenced at age 10 years at a dose of 10 mg po BID.

Once tyrosinemia I was established biochemically, the need for a liver biopsy was less pressing as the clinical status was assessed on the basis of liver function parameters and, later, the response to NTBC. However, hepatic imaging studies, which included magnetic resonance, ultrasound, and computerized tomography, uniformly document marked parenchymal inhomogeneity, reflective of fibrosis with regenerative nodules, or focal lesions in a cirrhotic liver (Rofsky et al., “CT and MRI of Diffuse Liver Disease,” Semin. Ultrasound CT MR, 16:16-33 (1995), which is hereby incorporated by reference in its entirety). Portal hypertension has evolved and is evidenced by splenomegaly and hypersplenism, as well as by collateral circulation and a caput medusa pattern demonstrated by Doppler ultrasound (Kok et al., “The Value of Doppler Ultrasound in Cirrhosis and Portal Hypertension,” Scand. J. Gastroenterol., 230(Suppl.):82-8 (1999), which is hereby incorporated by reference in its entirety).

### Example 3

### Computational Analysis of Molecules

The sequences for 4-HPPD of H. sapiens (Accession #AAC73008) and P. fluorescens (Accession #ICIXA) were aligned using the ClustalW algorithm of MacVector (Oxford Molecular, Madison, Wis.) to identify the active site segments and quantify their degree of conservation. The only publicly available crystal structure coordinates for 2-oxoacid-utilizing dioxygenases were downloaded from the PDB database under locator ICIX [4-HPPD of P. fluorescens] (Serre et al., “Crystal Structure of Pseudomonas Fluorescens 4-Hydroxyphenylpyruvate Dioxygenase: An Enzyme Involved in the Tyrosine Degradation Pathway,” Structure 7:977-88 (1999), which is hereby incorporated by reference in its entirety), and under locator IRXG [deactoxycephalosporin C synthase of S. clavuligerus] (Valegard et al., “Structure of a Cephalosporin Synthase,” Nature 394:805-9 (1998), which is hereby incorporated by reference in its entirety); the latter provides the representative coordinates for the alignment of the atmospheric oxygen and the 2-oxoacid substrates at the non-heme iron of these enzymes. Three-dimensional images of the active site of 4-HPPD were developed with the MSI InsightII software package (Molecular Simulations Inc., San Diego, Calif.). The 4-HPPD substrate 4-hydroxyphenylpyruvate (4-HPP) and the 4-HPPD inhibitor 2-(2-nitro-4-trifluormethylbenzoyl)-cyclohexane-1,3-dione (NTBC) were modeled and optimized using MacSpartan Plus (Wavefunction Inc., Irvine, Calif.), and then exported as PDB files into MSI InsightII.

It is well-established that the in vivo activity of the 2-oxoacid utilizing dioxygenases 4-HPPD and P4-H, which both use the HAG mechanism for catalysis, is directly reflected by their specific biomarkers. To assess 4-HPPD activity, two deleterious metabolites of the enzyme's product homogentisic acid, i.e., succinylacetone and succinylacetoacetate, have been measured in biologic fluids (Lindstedt et al., “Treatment of Hereditary Tyrosinemia Type I by Inhibition of 4-Hydroxyphenylpyruvate Dioxygenase,” Lancet 340:813-17 (1992); Holme et al., “Tyrosinemia Type I and NTBC [2-(2-Nitro-4-Trifluoromethylbenzoyl)-1,3-Cyclohexanedione],” J. Inherit Metab. Dis., 21:507-17 (1998), which are hereby incorporated by reference in their entirety). To assess P4-H activity, several collagen-derived antigens have been employed. Since all collagen molecules depend on P4-H-synthesized hydroxyproline residues to stabilize their triple helix (Bella et al., “Crystal and Molecular Structure of a Collagen-Like Peptide at 1.9 Å Resolution,” Science 266:75-81 (1994), which is hereby incorporated by reference in its entirety), inhibition of P4-H renders helix formation impossible (Hanauske-Abel, “Fibrosis: Representative Molecular Elements, A Basic Concept, and Emerging Targets for Suppressive Treatment,” in Zakim, eds. Hepatology, A Textbook of Liver Disease,” Philadelphia: W.B. Saunders, pp. 465-506 (1996); Hanauske-Abel, “Prolyl 4-Hydroxylase, A Target Enzyme for Drug Development. Design of Suppressive Agents and the In Vitro Effects of Inhibitors and Proinhibitors,” J. Hepatol., 13(Suppl. 3):S8-S16 (1991), which are hereby incorporated by reference in their entirety). In the case of fibrillar collagens like type III, the underhydroxylated non-helical material is retained and degraded intracellularly (Tschank et al., “Pyridine-Dicarboxylates, the First Mechanism-Derived Inhibitors for Prolyl 4-Hydroxylase, Selectively Suppress Cellular Hydroxyproline Biosynthesis,” Biochem J. 248:625-33 (1987), which is hereby incorporated by reference in its entirety). P4-H inhibition in cell culture (McCaffrey et al., “Specific Inhibition of eIF-5A and Collagen Hydroxylation by a Single Agent: Antiproliferative and Fibro-Suppressive Effects on Smooth Muscle Cells from Human Coronary Arteries,” J. Clin. Invest. 95:446-55 (1995), which is hereby incorporated by reference in its entirety) or in patients (Hanauske-Abel, “Fibrosis: Representative Molecular Elements, A Basic Concept, and Emerging Targets for Suppressive Treatment,” in Zakim, eds. Hepatology, A Textbook of Liver Disease,” Philadelphia: W.B. Saunders, pp. 465-506 (1996); Newfield et al., “Deferiprone, but not Deferoxamine, Inhibits Biosynthesis of Fibrillar Procollagen in Iron-Overloaded Thalassemia Patients,” Pediatr. Res. 39:A943 (1996), which are hereby incorporated by reference in their entirety) causes a marked and rapid decrease in collagen biomarkers levels. In the case of sheet-forming collagens like type IV, the under-hydroxylated non-helical material may be secreted, whereupon it fails to incorporate into matrix and is degraded extracellularly (Kim et al., “Differential Effects of Ascorbate Depletion and Alpha, Alpha-Dipyridyl Treatment on the Stability, but not on the Secretion, of Collagen Type IV Collagen in Differentiated P9 Cells,” J. Cell Biochem. 67:338-52 (1997); Frei et al., “The N-Terminal Propeptide of Collagen Type III in Serum Reflects Activity and Degree of Fibrosis in Patients with Chronic Liver Disease,” Hepatology 4:830-834 (1984), which are hereby incorporated by reference in their entirety). These collagen type-specific consequences of P4-H inhibition suggested the use of several, collagen-type specific biomarkers, i.e., of NCI and NTD in addition to PIIINP. The serum concentration of each of these biomarkers, derived from collagens type III, type IV, and type VI, respectively, also is known to be directly related to, and markedly increases with, fibrotic liver disease (Murawaki et al., “Comparison of Serum 7S Fragment of Type IV Collagen and Serum Central Triple-Helix of Type IV Collagen for Assessment of Liver Fibrosis in Patients with Chronic Viral Liver Disease,” J. Hepatol. 24:148-54 (1996); Pitkanen et al., “Serum Type III Procollagen in Children with Type I Hereditary Tyrosinemia,” J. Ped. Gastroenterol. Nutr. 29:38-41 (1999), which are hereby incorporated by reference in their entirety). The P1 antigen of laminin, the major matrix protein that lacks hydroxyproline residues and in culture is secreted normally even as P4-H is completely inhibited (Kim et al., “Differential Effects of Ascorbate Depletion and Alpha, Alpha-Dipyridyl Treatment on the Stability, but not on the Secretion, of Collagen Type IV Collagen in Differentiated P9 Cells,” J. Cell Biochem. 67:338-52 (1997), which is hereby incorporated by reference in its entirety), was measured as control parameter.

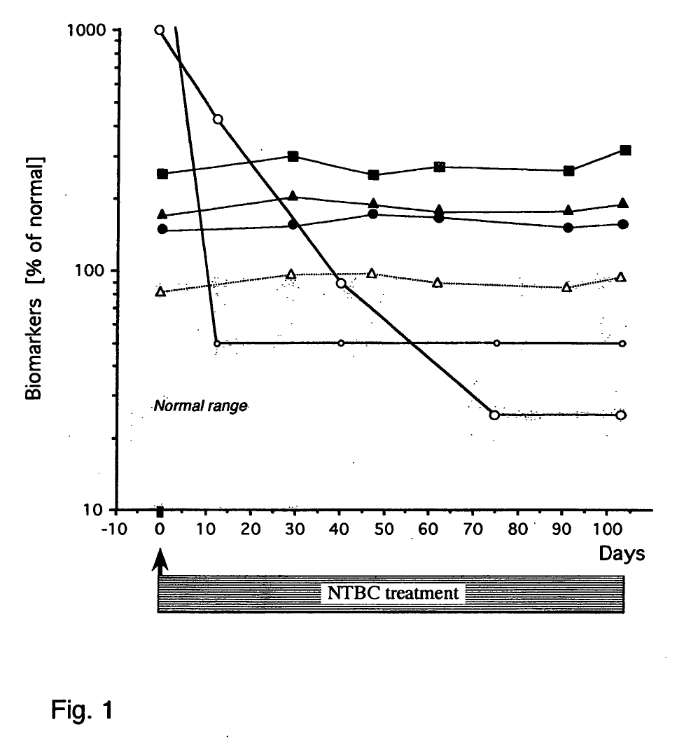

In the tyrosinemic patient, the biomarkers for 4-HPPD activity promptly decreased upon initiation of NTBC treatment. Initially elevated to more than ten-fold its physiological level, the excretion of succinylacetoacetate normalized within two weeks. The grossly increased plasma concentration of succinylacetone also fell, although more gradually, and stabilized in the normal range after two months of treatment (FIG. 1). These observations are consistent with the previously reported effect of NTBC on these biomarkers for the activity of human 4-HPPD, and confirm the distinctly different kinetics of their response (Lindstedt et al., “Treatment of Hereditary Tyrosinemia Type I by Inhibition of 4-Hydroxyphenylpyruvate Dioxygenase,” Lancet 340:813-17 (1992), which is hereby incorporated by reference in its entirety).

The biomarkers for P4-H activity were all entirely unresponsive to NTBC, however (FIG. 1). The levels of PIIINP, NC1, and NTD, documented before initiation of treatment, ranged at the minimum 1.6 multiples above the age-related medians, and as such revealed an active and on-going fibrogenic process in the patient's liver (Murawaki et al., “Comparison of Serum 7S Fragment of Type IV Collagen and Serum Central Triple-Helix of Type IV Collagen for Assessment of Liver Fibrosis in Patients with Chronic Viral Liver Disease,” J. Hepatol. 24:148-54 (1996); Pitkanen et al., “Serum Type III Procollagen in Children with Type I Hereditary Tyrosinemia,” J. Ped. Gastroenterol. Nutr. 29:38-41 (1999), which are hereby incorporated by reference in their entirety). These initially abnormal levels remained consistently elevated throughout the three months of the study period and were unaffected by the administration of NTBC, although in humans, effective P4-H inhibition causes the biomarkers for at least the fibrillar collagens to decrease within a few days (Hanauske-Abel, “Fibrosis: Representative Molecular Elements, A Basic Concept, and Emerging Targets for Suppressive Treatment,” in Zakim, eds. Hepatology, A Textbook of Liver Disease,” Philadelphia: W.B. Saunders, pp. 465-506 (1996); Newfield et al., “Deferiprone, but not Deferoxamine, Inhibits Biosynthesis of Fibrillar Procollagen in Iron-Overloaded Thalassemia Patients,” Pediatr. Res. 39:A943 (1996), which are hereby incorporated by reference in their entirety). In fact, all the biomarkers for collagenous and, therefore, P4-H-dependent proteins (i.e., PIIINP, NC1, and NTD) were consistently just as unresponsive to NTBC treatment as the non-collagenous and, therefore, P4-H independent protein laminin, assessed via its P1 antigen (FIG. 1). After 103 days on NTBC, all the biomarkers for 4-HPPD activity had normalized, averaging 3% of their initial values, whereas the PIIINP biomarker for P4-H activity displayed a distinct increase to 1.25 times its pre-NTBC level, or three times the upper limit of normal. The other P4-H biomarkers also did not just fail to decrease, but showed a similar, though smaller rise over their initial, abnormally elevated values, NCI and NTC both increasing by a factor of 1.1. After 273 days on NTBC, PIIINP had increased even further, to 1.35 times its value at treatment initiation.

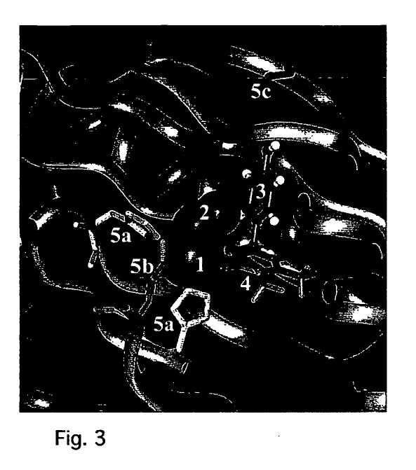

While applicants' investigation was in progress, a brief report described in one other tyrosinemic child that the initially elevated PIIINP level was non-responsive to treatment with NTBC (Pitkanen et al., “Serum Type III Procollagen in Children with Type I Hereditary Tyrosinemia,” J. Ped. Gastroenterol. Nutr. 29:38-41 (1999), which is hereby incorporated by reference in its entirety). This case observation and the applicants' results, summarized in FIG. 1, indicate that in vivo, NTBC affects the 2-oxoacid utilizing dioxygenases 4-HPPD and P4-H in an entirely different manner, all the biomarkers for the former being highly susceptible, and all those for the latter totally resistant to inhibition. Using an unspecified in vitro assay for P4-H activity isolated from an unidentified species, the developers of NTBC cursorily mentioned a similar observation (Ellis et al., “Inhibition of 4-Hydroxyphenylpyruvate Dioxygenase by 2-(2-Nitro-4-Trifluormethylbenzoyl)-Cyclohexane-1,3-Dione and by 2-(2-Chloro-4-Methanesulfonylbenzoyl)-Cyclohexane-1,3-Dione,” Toxicol. Appl. Pharmacol. 133:12-19 (1995), which is hereby incorporated by reference in its entirety). Together, these repeatedly noted differences indicate that the inhibitor NTBC only affects the toxogenic activity of 4-HPPD in tyrosinemic patients, but does not affect the fibrogenic activity of prolyl 4-hydroxylase, although both enzymes belong to the same class of dioxygenases and follow the same catalytic mechanism. The patients thus remain vulnerable to long-term liver failure due to the slowly progressing fibrosis and evolving cirrhosis. By contrast, compounds of the present invention as embodied by DMHP are known inhibitors of prolyl 4-hydroxylase and, therefore, collagen biosynthesis (U.S. Pat. Nos. 5,789,426; 5,965,585; and 5,965,586, which are hereby incorporated by reference in their entirety), and also fit the active site of 4-HPPD, thus blocking the enzyme's toxogenic activity in these patients in a manner similar to NTBC (see FIG. 3).

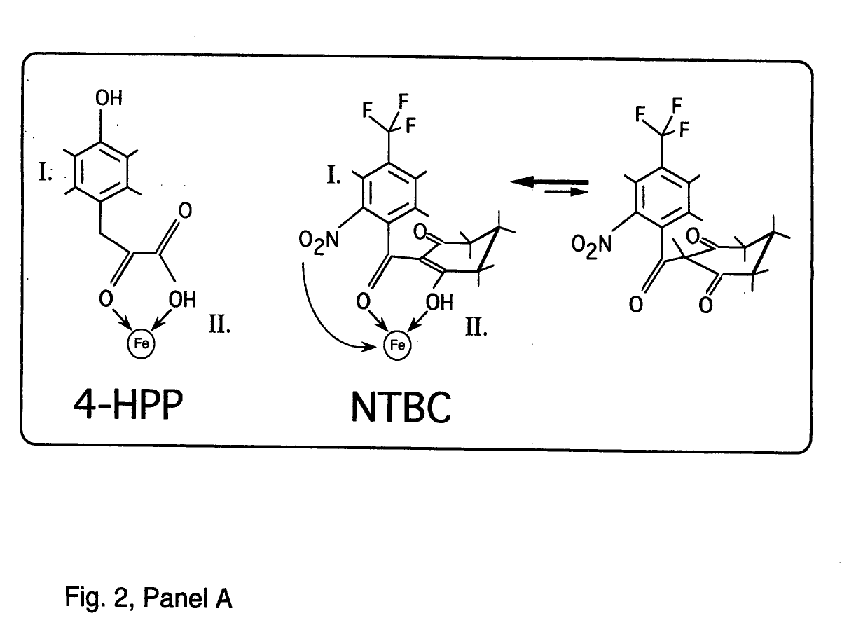

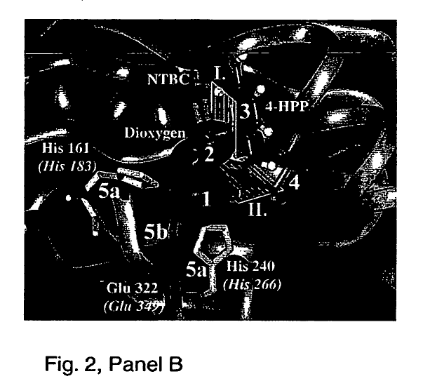

The stereochemistry of substrate and inhibitor binding to P4-H is known (Hanauske-Abel, “Prolyl 4-Hydroxylase, A Target Enzyme for Drug Development. Design of Suppressive Agents and the In Vitro Effects of Inhibitors and Proinhibitors,” J. Hepatol., 13(Suppl. 3):S8-S16 (1991), which is hereby incorporated by reference in its entirety). The stereochemistry of substrate and inhibitor binding to 4-HPPD, though highly relevant for the current state-of-the-art treatment of tyrosinemia I, has not been formulated, yet it is evident from crystallographic and computational data, as summarized in FIG. 2. Consistent with the HAG mechanism (Hanauske-Abel et al., “A Stereochemical Concept for the Catalytic Mechanism of Prolyl Hydroxylase. Applicability to Classification and Design of Inhibitors,” J. Theor. Biol. 94:421-55 (1982); Hanauske-Abel, “Fibrosis: Representative Molecular Elements, A Basic Concept, and Emerging Targets for Suppressive Treatment,” in Zakim, eds. Hepatology, A Textbook of Liver Disease,” Philadelphia: W.B. Saunders, pp. 465-506 (1996), which are hereby incorporated by reference in their entirety), the enzyme's substrate 4-hydroxyphenylpyruvate (4-HPP) displays two domains, one defined by the benzene ring (domain I) and one by the 2-oxoacid group (domain II); the same domain structure applies to NTBC (FIG. 2A). In both agents, these domains define planes that are markedly out-of-level with each other (FIG. 2B). In the case of 4-HPP, domain I is rotated by almost 70° above domain II and oriented towards the binding site of dioxygen, the source of the reactive oxygen atom that, in the product-forming step, transfers from the iron onto the benzene ring (Abbott et al., “Alpha-Ketoglutarate-Coupled Dioxygenases,” in Hayaishi, ed. Molecular Mechanisms of Oxygen Activation, New York: Academic Press, pp. 167-214 (1974); Hanauske-Abel et al., “A Stereochemical Concept for the Catalytic Mechanism of Prolyl Hydroxylase. Applicability to Classification and Design of Inhibitors,” J. Theor. Biol. 94:421-55 (1982), which are hereby incorporated by reference in their entirety). In the case of NTBC, domain I also is oriented towards that same binding site, which places the electronegative nitro group precisely into the position usually occupied by the electronegative dioxygen (FIG. 2B). This computer-generated alignment suggests that the ortho-positioned nitro group can, in a dioxygen-like manner, directly interact with the active site iron, an effect that at the active site of the enzyme would make NTBC a terdentate ligand, in contrast to the bidentate coordination of 4-HPP (FIG. 2). Indeed, experimental studies of the structure-activity relation for NTBC and similar cyclohexanetrionics confirm that the presence of an ortho-positioned electronegative group, carried by a properly oriented benzene ring, is important for the effective inhibition of 4-HPPD (Lin et al. “SAR Studies of 2-O-substituted-benzoyl- and 2-alkanoyl-cyclohexane-1,3-diones as Inhibitors of 4-hydroxyphenylpyruvate dioxygenases,” Bioorg. Med. Chem. Lett. 10:843-45 (2000), which is hereby incorporated by reference in its entirety). In applicants' analysis, the ability to coordinate to, and thus block, the catalytically essential non-heme iron atom emerges as an important feature of NTBC binding to the active site of 4-HPPD. This pivotal conclusion was subsequently corroborated by independent investigators (Wu et al. “Mode of Action of 4-hydroxyphenylpyruvate dioxygenase Inhibition by Triketone-type Inhibitors,” J. Med. Chem. 45:2222-28 (2002), which is hereby incorporated by reference in its entirety). This ability is a general characteristic of effective inhibitors for other 2-oxoacid-utilizing dioxygenases, in particular P4-H (Hanauske-Abel, “Prolyl 4-Hydroxylase, A Target Enzyme for Drug Development. Design of Suppressive Agents and the In Vitro Effects of Inhibitors and Proinhibitors,” J. Hepatol., 13(Suppl. 3):S8-S16 (1991); Günzler et al., “Prolyl 4-Hydroxylase Inhibitors,” in Guzman N A ed., Prolyl Hyroxylase, Protein Disulfide Isomerase, and Other Structurally Related Proteins, New York: Decker, pp. 65-96 (1997); Ng et al., “The Cosubstrate Binding Site of Pseudomonas sp. AK1 γ-Butyrobetaine Hydroxylase: Interactions with Structural Analogs of α-Ketoglutarate,” J. Biol. Chem. 266:1526-33 (1991), which are hereby incorporated by reference in their entirety). However, the active site of P4-H requires in addition to this general characteristic, that domain I is positioned in the plane of domain II, rendering P4-H particularly susceptible to entirely planar inhibitors (Hanauske-Abel, “Über einen stereochemischen Vorschlag für den katalytischen Mechanismus der Prolylhydroxylase, seine Anwendung zur Klassifizierung und Formulierung von Hemmstoffen sowie die Entwicklung und Erprobung eines ‘maβgeschneiderten’ neuartigen Inhibitors,” Department of Pharmacol. Toxicol., Philipps University, Marburg an der Lahn, Germany, M.D., Ph.D., Thesis (1983); Günzler et al., “Prolyl 4-Hydroxylase Inhibitors,” in Guzman N A ed., Prolyl Hyroxylase, Protein Disulfide Isomerase, and Other Structurally Related Proteins, New York: Decker, pp.65-96 (1997); Ng et al., “The Cosubstrate Binding Site of Pseudomonas sp. AK1 γ-Butyrobetaine Hydroxylase: Interactions with Structural Analogs of α-Ketoglutarate,” J. Biol. Chem. 266:1526-33 (1991), which are hereby incorporated by reference in their entirety). The out-of-level alignment of domain I-vs.-domain II, as visualized in FIG. 2 for 4-HPPD, is in distinct contrast to the conformation of all known inhibitors for P4-H. 4-HPPD and P4-H should, therefore, display markedly different susceptibilities to NTBC. As shown in FIG. 1, this is indeed the case. In contrast to NTBC, the compounds of the present invention as embodied by DMHP lack any structural equivalent of domain I (compare FIG. 2B with FIG. 3) and therefore, are not subject to the biological and clinical consequences of misalignment between these domains. In fact, DMHP so closely mimics the dimensions and the planarity of domain II of the 4-HPP substrate that both entities assume isomorphic positioning at the active site of 4-HPPD, as shown in FIG. 3. The structure-based approach of the present invention also is applicable to the evaluation of agents other than DMHP or NTBC. For instance, usnic acid, a metabolite produced by lichen to suppress competing plants (Moradpur et al., “Hepatic Oncogenesis,” in Zakim, eds., Hepatology, Philadelphia: Saunders, pp. 1490-1512 (1996), which is hereby incorporated by reference in its entirety) and an ingredient in over-the-counter dietary supplements, is a potent inhibitor of plant 4-HPPD (Romagni et al., “The Phytotoxic Lichen Metabolite, Usnic Acid, is a Potent Inhibitor of Plant p-hydroxyphenylpyruvate dioxygenase,” FEBS Lett. 480:301-05 (2000), which is hereby incorporated by reference in its entirety) and fits the 4-HPPD model presented in FIG. 2. This provides a rationale for the clinical evaluation of usnic acid as a 4-HPPD inhibitor in tyrosinemia I. In general, computer-assisted modeling has been extraordinarily successful in guiding the development of inhibitors for several enzymes in the class of 2-oxoacid utilizing dioxygenases (Hanauske-Abel et al., “The HAG Mechanism: A Molecular Rationale for the Therapeutic Application of Iron Chelators in Human Diseases Involving the 2-oxoacid Utilizing Dioxygenases,” Curr. Med. Chem. 10:1005-19 (2003), which is hereby incorporated by reference in its entirety).

The persistent elevation of several indicators for hepatic fibrogenesis suggests that even after NTBC medication achieves biochemical normalization of the toxic metabolites of 4-HPPD in tyrosinemia I, the biologic correction of the fibrotic process does not occur readily (FIG. 1; also Pitkanen et al., “Serum Type III Procollagen in Children with Type I Hereditary Tyrosinemia,” J. Ped. Gastroenterol. Nutr. 29:38-41 (1999), which is hereby incorporated by reference in its entirety). This caveat is supported by the histopathologic evaluation of repeated liver biopsies performed on patients with tyrosinemia I before and during prolonged treatment with NTBC. The abnormal hepatic metabolism of several major collagens, sustained at a markedly increased level, should raise the concerns that over the lifetime of these children, a fibrotic process even if low-grade may eventually lead to cirrhosis and long-term organ failure, and may figure as one factor in the development of hepatocellular carcinoma. The latter, which occurs in 40% of tyrosinemic patients not treated with NTBC (Moradpur et al., “Hepatic Oncogenesis” In Hepatology, D. Zakim and T. D. Boyer, ed. Saunders, Philadelphia. 1490-1512 (1996), which is hereby incorporated by reference in its entirety), has been reported during the first year (Holme et al., “Nontransplant Treatment of Tyrosinemia,” Clin. Liver Dis. 4:805-814 (2000), which is hereby incorporated by reference in its entirety) and the second year (Viladoms et al., “Evolution of Tyrosinemia Type I Treated with NTBC,” An Esp. Pediatr. 54:305-9 (2001), which is hereby incorporated by reference in its entirety) of treatment with NTBC. While early treatment with NTBC may decrease the incidence of this serious complication (Holme et al., “Tyrosinemia Type I and NTBC [2-(2-Nitro-4-Trifluoromethylbenzoyl)-1,3-Cyclohexanedione],” J. Inherit Metab. Dis., 21:507-17 (1998); Russo et al., “Tyrosinemia: A Review,” Pediatr. Dev. Pathol. 4:212-221 (2001), which are hereby incorporated by reference in their entirety), it remains entirely unknown at present whether treatment with NTBC affects the evolving liver fibrosis and its sequelae, such as portal hypertension, which like hepatocellular carcinoma rank among the major issues in the care for these children (Russo et al., “Tyrosinemia: A Review,” Pediatr. Dev. Pathol. 4:212-221 (2001), which is hereby incorporated by reference in its entirety). By contrast, in experimental animals, the targeted inhibition of P4-H, which markedly suppresses hepatic fibrosis, limits both the development of portal hypertension (Hanauske-Abel, “Fibrosis: Representative Molecular Elements, A Basic Concept, and Emerging Targets for Suppressive Treatment,” in Zakim, eds. Hepatology, A Textbook of Liver Disease,” Philadelphia: W.B. Saunders, pp. 465-506 (1996), which is hereby incorporated by reference in its entirety) and of hepatic neoplasms (Sakaida et al., “Prevention of Fibrosis Reduces Enzyme-altered Lesions in the Rat Liver,” Carcinogenesis 15:2201-06 (1994), which is hereby incorporated by reference in its entirety). Joint inhibition of 4-HPPD and P4-H, or inhibition of just P4-H, would be particularly reassuring for these patients, and would also provide an invaluable inroad into the development of P4-H inhibitors for use as hepatoselective fibrosuppressors (Hanauske-Abel, “Fibrosis: Representative Molecular Elements, A Basic Concept, and Emerging Targets for Suppressive Treatment,” in Zakim, eds. Hepatology, A Textbook of Liver Disease,” Philadelphia: W.B. Saunders, pp. 465-506 (1996); Hanauske-Abel, “Prolyl 4-Hydroxylase, A Target Enzyme for Drug Development. Design of Suppressive Agents and the In Vitro Effects of Inhibitors and Proinhibitors,” J. Hepatol., 13(Suppl. 3):S8-S16 (1991), which are hereby incorporated by reference in their entirety), compounds of general interest in treatment of fibrotic liver disease. Applicants' computational analysis of agents that already are clinically available, suggests that hydroxypyridone compounds not only inhibit P4-H (U.S. Pat. No. 5,965,586 to Hanauske-Abel et al. and Clement et al., “The Antifungal Drug Ciclopirox Inhibits Deoxyhypusine and Proline Hydroxylation, Endothelial Cell Growth, and Angiogenesis In vitro,” Int'l J. Cancer 100:491-98 (2002), which are hereby incorporated by reference in their entirety) and selectively suppress collagen formation (U.S. Pat. No. 5,789,426 to Hanauske-Abel et al., which is hereby incorporated by reference in its entirety), but also fit the active site of 4-HPPD. As exemplified in FIG. 3 by DMHP, hydroxypyridones locate precisely into the van der Waals volume occupied at the active site by the cyclohexane-1,3-dione moiety of NTBC and its analogs. It is, therefore, evident that the hydroxypyridone inhibitors of P4-H will also inhibit 4-HPPD in vitro and in vivo, will be useful as pilot structures for chemical optimization, and represent promising candidates for clinical trials.

Finally, in addition to the 3D coordinate of 4-HPPD, the crystal structures of several additional 2-oxoacid-utilizing dioxygenases have been deposited in the PDB database (Berman et al. “The Protein Data Bank” Nucl. Acids Res. 28:235-42 (2000), which is hereby incorporated by reference in its entirety). Those coordinates for 4-HPPD are at locator 1CJX and have been used by the applicants. Currently, crystal structures are available for the following enzymes that also employ the HAG mechanism: clavaminate synthase (locators 1DS0, 1DS1, 1DRT, 1DRY); carbapenem synthase (locators 1NX8, 1NX4); deactoxycephalosporin C synthase (locators 1RXG, 1RXF, 1E5I, 1E5H); anthocyanidin synthase (locators 1GP4, 1GP5, 1GP6); proline 3-hydroxylase (locator 1E5R, 1E5S); taurine dioxygenase (1GY9, 1GQW); and the first representative example for the sub-family of protein hydroxylases, FIH1, one of the enzymes which modify and control hypoxia inducible factor-1 alpha (locators 1H2K, 1H2L, 1H2M, 1H2N, 1MZF). The active site coordinates of all these enzymes reveal the same cisoid coordination of a non-heme ferrous ion, with the same relative orientation of two histidines and one carboxyl side chain (compare FIGS. 2 and 3) imposed by the HAG mechanism (Hanauske-Abel et al., “The HAG Mechanism: A Molecular Rationale for the Therapeutic Application of Iron Chelators in Human Diseases Involving the 2-Oxoacid Utilizing Dioxygenases,” Curr. Med. Chem. 10:1005-19 (2003), which is hereby incorporated by reference in its entirety). This atomic arrangement at the active site of all enzymes of this class, summarized in Table III, also provides the coordination sites at the catalytic ferrous ion that are required for interaction with, and inhibition by, the hydroxypyridone compounds of the present invention.

TABLE III
Representative examples for HAG dioxygenases and their biological functions.*

| Enzyme | Dependent biological event | Species |
|---|---|---|
| Prolyl 4-hydroxylase (I and II) | Synthesis of collagens and collectins | H, A |
| HIF Prolyl 4-hydroxylase (PHD1-3) | Oxygen-sensing via hypoxia-inducible | H, A |
| | factor | |
| Aspartyl/asparaginyl 3-hydroxylase | Synthesis of fibrillins and LTBPs | H, A |
| | Bioactivity of TGF-βs | |
| HIF Aspartyl/asparaginyl 3-hydroxylase (FIH- | Oxygen-sensing via hypoxia-inducible | H |
| 1) | factor | |
| Deoxyhypusyl hydroxylase | Control of G1-to-S transition in the cell | H, A, Pl, |
| | cycle, i.e. initiation of DNA replication; | fungi and |
| | Multiplication of retroviruses, e.g. HIV-1 | archaea |
| Lysyl 5-hydroxylase 1 to 3 | Crosslinking/glycosylation of collagens | H, A |
| 4-Hydroxyphenylpyruvate dioxygenase | Tyrosine catabolism Tyrosine catabolism, | H, A, M Pl |
| apparently identical with | antioxidant synthesis Plastochinone/ | |
| α-Ketoisocaproate oxygenase | tocopherol synthesis | |
| | Leucine catabolism | H, A |
| AlkB protein | Reversal of DNA alkylation | H M |
| ε-N-Trimethyllysine hydroxylase | Carnitine formation | H, A, M |
| γ-Butyrobetaine hydroxylase | Carnitine formation | H, A, M |
| Phytanoyl-CoA 2-hydroxylase | Phytanic acid metabolism | H |
| Hyoscyamine 6β-hydroxylase | Atropine/scopolamine synthesis | Pl |
| Desacetoxyvindoline 4-hydroxylase | Vincristine/vinblastine synthesis | Pl |
| Anthocyanidin synthase | Anthocyanin and related plant colors | Pl |
| 2,4-dichlorophenoxyacetic acid dioxygenase | Herbicide metabolism | Pl |
| Isoleucine 4-hydroxylase | Unknown | Pl |
| Flavone synthase I | Flavone synthesis, nodulation | Pl |
| (2S)-Flavanone 3β-hydroxylase | Catechin/anthocyanidin synthesis | Pl |
| Flavanol 6-hydroxylase | Flavanol synthesis | Pl |
| Gibberellin 2β-hydroxylase | Plant growth | Pl |
| Gibberellin 3β-hydroxylase | Plant growth | Pl |
| Gibberellin 20-hydroxylase | Plant growth | Pl |
| Prolyl 4-hydroxylase | Cell wall synthesis, defense | Pl |
| Proline 4-hydroxylase | Actinomycin synthesis | M |
| Proline 3-hydroxylase | 3-hydroxyproline formation | M |
| Thymidine 2′-hydroxylase | Nucleotide salvage | M |
| Thymine 7-hydroxylase | Thymine catabolism | M |
| Taurine dioxygenase | Sulfur metabolism | M |
| 2-hydroxymyristate dioxygenase | Lipid A/endotoxin synthesis | M |
| Deactoxycephalosporin C synthase | Cephalosporin synthesis | M |
| Deactoxycephalosporin C hydroxylase (3′- | Cephalosporin synthesis | M |
| methylcephem hydroxylase) | | |
| Deactoxycephalosporin C synthase/hydroxylase | Cephalosporin synthesis | M |
| Cephalosporin 7α-hydroxylase | Cephamycin synthesis | M |
| Clavaminate synthase | β-Lactamase inhibitor synthesis | M |

Abbreviations: Humans (H); animals (A); plants (Pl): microorganisms (M)
*See, e.g., Hanauske-Abel et al., “The HAG Mechanism: A Molecular Rationale For the Therapeutic Application of Iron Chelators in Human Diseases Involving the 2-Oxoacid Utilizing Dioxygenases,” Curr. Med. Chem. 10: 1005-19 (2003), which is hereby incorporated by reference in its entirety.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

1. A method of inhibiting 4-hydroxyphenylpyruvate dioxygenase in a living system comprising:
administering to the living system an effective amount of a compound of formulas I or II or III or derivatives thereof as follows: 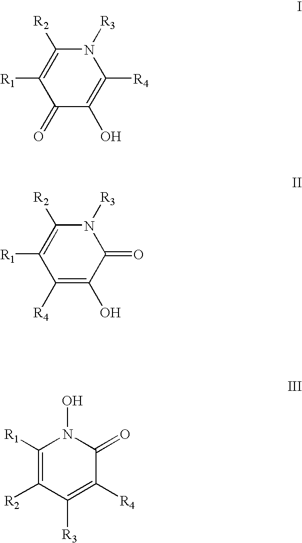
R¹, R², R³, and R⁴ each individually represent a hydrogen, an alkyl, alkenyl, or alkoxy group containing 1 to about 8 carbon atoms, an aryl, aralkyl, or cycloalkyl group containing about 5 to 12 carbon atoms, or a carboalkoxy or carbamyl group containing up to 8 carbon atoms, or a peptide or peptidomimetic moiety containing 10 to about 30 carbon atoms.

2. A method according to claim 1, wherein the compound is deferiprone (DMHP).

3. A method of treating a patient with tyrosinemia comprising:
administering to the patient an effective amount of a compound of formulas I or II or III or derivatives thereof as follows: 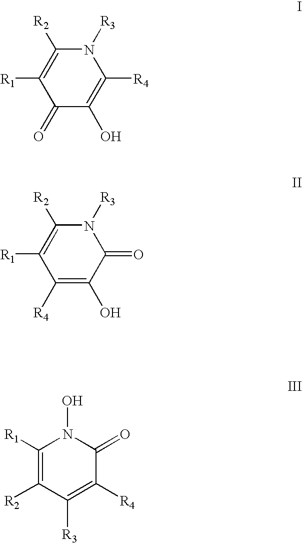
R¹, R², R³, and R⁴ each individually represent a hydrogen, an alkyl, alkenyl, or alkoxy group containing 1 to about 8 carbon atoms, an aryl, aralkyl, or cycloalkyl group containing about 5 to 12 carbon atoms, or a carboalkoxy or carbamyl group containing up to 8 carbon atoms, or a peptide or peptidomimetic moiety containing 10 to about 30 carbon atoms.

4. A method according to claim 3, wherein the compound is deferiprone (DMHP).

5. A method of regulating plant growth comprising:
administering to a plant an effective amount of a compound of formulas I or II or III or derivatives thereof as follows: 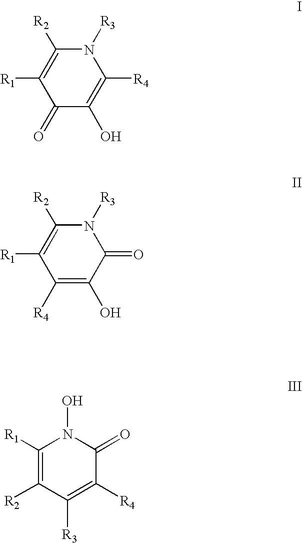
R¹, R², R³, and R⁴ each individually represent a hydrogen, an alkyl, alkenyl, or alkoxy group containing 1 to about 8 carbon atoms, an aryl, aralkyl, or cycloalkyl group containing about 5 to 12 carbon atoms, or a carboalkoxy or carbamyl group containing up to 8 carbon atoms, or a peptide or peptidomimetic moiety containing 10 to about 30 carbon atoms.

6. A method according to claim 5, wherein the compound is deferiprone (DMHP).
